(19) United States
(12) Patent Application Publication
(10) Pub. No.: US 2008/0319523 A1
(43) Pub. Date: Dec. 25, 2008

(54) ANEURYSM FILLER DEVICE

(75) Inventor: Christina Young, Maple Grove, MN (US)

Correspondence Address: SCHWEGMAN, LUNDBERG & WOESSNER, P.A., P.O. BOX 2938, MINNEAPOLIS, MN, 55402, US

(73) Assignee: Neuro Vasx, Inc, North Maple Grove, MN (US)

(21) Appl. No.: 11/767,300

(22) Filed: Jun. 22, 2007

(51) Int. Cl. A61F 2/06

(52) U.S. Cl. 623/1.11

(57) ABSTRACT

Embodiments of the invention include an aneurysm filler devide comprising: a main body comprising a pusher portion, a filler portion and a bridge portion, wherein the bridge portion contacts both the pusher portion and the filler portion.

### FIELD

The inventive subject matter described herein relates to an aneurysm filler device wire and to a method for repairing an aneurysm. The inventive subject matter also relates to a method for making, a method for using and to a method for detaching an aneurysm filler device.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the products, processes and data as described below and in the tables that form a part of this document: Copyright 2007, Neurovasx, Inc. All Rights Reserved.

### BACKGROUND OF THE INVENTION

An aneurysm is a balloon-like swelling in a wall of a blood vessel. Aneurysms result in weakness of the vessel wall in which it occurs. This weakness predisposes the vessel to tear or rupture with potentially catastrophic consequences for any individual having the aneurysm. Vascular aneurysms are a result of an abnormal dilation of a blood vessel, usually resulting from disease and/or genetic predisposition which can weaken the arterial wall and allow it to expand. Aneurysm sites tend to be areas of mechanical stress concentration so that fluid flow seems to be the most likely initiating cause for the formation of these aneurysms.

Aneurysms in cerebral circulation tend to occur in an anterior communicating artery, posterior communicating artery, and a middle cerebral artery. The majority of these aneurysms arise from either curvature in the vessels or at bifurcations of these vessels. The majority of cerebral aneurysms occur in women. Cerebral aneurysms are most often diagnosed by the rupture and subarachnoid bleeding of the aneurysm.

Cerebral aneurysms are most commonly treated in open surgical procedures where the diseased vessel segment is clipped across the base of the aneurysm. While considered to be an effective surgical technique, particularly considering an alternative which may be a ruptured or re-bleed of a cerebral aneurysm, conventional neurosurgery suffers from a number of disadvantages. The surgical procedure is complex and requires experienced surgeons and well-equipped surgical facilities. Surgical cerebral aneurysm repair has a relatively high mortality and morbidity rate of about 2% to 10%.

Current treatment options for cerebral aneurysm fall into two categories, surgical and interventional. The surgical option has been the long held standard of care for the treatment of aneurysms. Surgical treatment involves a long, delicate operative procedure that has a significant risk and a long period of postoperative rehabilitation and critical care. Successful surgery allows for an endothelial cell to endothelial cell closure of the aneurysm and therefore a cure for the disease. If an aneurysm is present within an artery in the brain and bursts, this creates a subarachnoid hemorrhage, and a possibility that death may occur. Additionally, even with successful surgery, recovery takes several weeks and often requires a lengthy hospital stay.

In order to overcome some of these drawbacks, interventional methods and prostheses have been developed to provide an artificial structural support to the vessel region impacted by the aneurysm. The structural support must have an ability to maintain its integrity under blood pressure conditions and impact pressure within an aneurysmal sac and thus prevent or minimize a chance of rupture. U.S. Pat. No. 5,405,379 to Lane, discloses a self-expanding cylindrical tube which is intended to span an aneurysm and result in isolating the aneurysm from blood flow. While this type of stent-like device may reduce the risk of aneurysm rupture, the device does not promote healing within the aneurysm. Furthermore, the stent may increase a risk of thrombosis and embolism. Additionally, the wall thickness of the stent may undesirably reduce the fluid flow rate in a blood vessel. Stents typically are not used to treat aneurysms in a bend in an artery or in tortuous vessels such as in the brain because stents tend to straighten the vessel.

U.S. Pat. No. 5,354,295 to Guglielmi et al., describes a type of vasoclusion coil. Disadvantages of use of this type of coil are that the coil may compact, may migrate over time, and the coil does not optimize the patient's natural healing processes.

### IN THE FIGURES

FIG. 1 illustrates an axial view of one embodiment of an aneurysm filler device.

FIG. 2 illustrates an axial view of another embodiment of an aneurysm filler device.

### DESCRIPTION

Although detailed embodiments of the invention are disclosed herein, it is to be understood that the disclosed embodiments are merely exemplary of the invention that may be embodied in various and alternative forms. Specific structural and functional details disclosed herein are not to be interpreted as limiting, but merely as a basis for teaching one skilled in the art to variously employ the aneurysm filler device embodiments. Throughout the drawings, like elements are given like numerals.

Referred to herein are trade names for materials including, but not limited to, polymers and optional components. The inventors herein do not intend to be limited by materials described and referenced by a certain trade name. Equivalent materials (e.g., those obtained from a different source under a different name or catalog (reference) number to those referenced by trade name may be substituted and utilized in the methods described and claimed herein. All percentages and ratios are calculated by weight unless otherwise indicated. All percentages are calculated based on the total composition unless otherwise indicated. All component or composition levels are in reference to the active level of that component or composition, and are exclusive of impurities, for example, residual solvents or by-products, which may be present in commercially available sources.

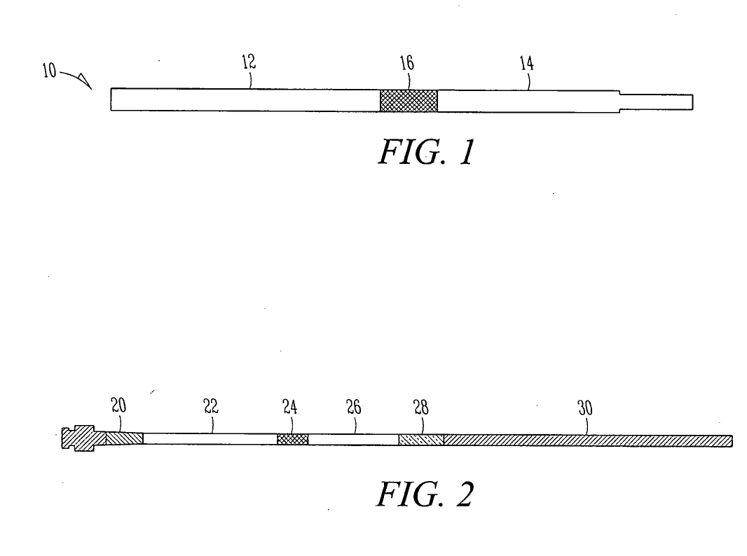

One embodiment of an aneurysm filler device, illustrated at 10 in FIG. 1, includes a pusher shaft 12, a distal filler portion 14 and a bridge 16 that contacts both the pusher shaft 12 and the distal filler portion 14. The bridge 16 has a compatibility with both the pusher shaft 12 and distal filler portion 14. The pusher shaft 12, in some embodiments is made of high density polyethylene, HDPE. The filler portion 14 is, for some embodiments, implant material such as 8445 Engage with 75% Tungsten, which are loaded into one or both polymeric materials that include HDPE and low density polyethylene, LDPE. For some embodiments, the components of the implant material are thermally welded, but have slightly different temperatures for melt processing.

The bridge 16 is made of any HDPE, LDPE, LLDPE, or other thermally compatible polyolefin. Because the bridge 16 is compatible with the pusher shaft 12 and distal filler portion 14, the bridge 16 may be welded to each of the pusher shaft 12 and distal filler portion 14, forming thermal bonds.

The aneurysm filler device embodiment 10 improves the ease by which the filler portion 30 can be delivered and added to an aneurysm, particularly, a brain aneurysm.

Another embodiment, shown at 20 in FIG. 2, illustrates an aneurysm filler device embodiment, that includes a first pusher shaft 22, a bridge 24, and a second distal pusher shaft 26, wherein the first pusher shaft 22 and second pusher shaft 26 both contact the bridge 24. The aneurysm filler device embodiment 20, also includes a second bridge 28 and filler portion 30, wherein both the second distal pusher shaft 25 and filler portion 30 contact the second bridge 28.

The aneurysm filler device embodiment 20 illustrates that the bridges 24 and 28 are usable to create a multi-directional aneurysm filler system by imparting improved control of movement of the aneurysm filler device embodiment. While embodiments are shown having one or two bridges, it is understood, that aneurysm filler detacher embodiments may have multiple bridges positioned along the length of the aneurysm filler device. The bridges impart stiffness transitions throughout the implant filler, and the pusher shaft, as needed or desired. For some embodiments, the position of one or more bridges improves pushability of the device. The position of the bridges also improves the flexibility of the device and provides variations in the implant material in order to optimize its filling capabilities.

The aneurysm filler material described herein may be one or more of polymeric and polymeric hybrids such as PEBAX, Grilamids, polyester, and silica. Materials also include reabsorbables such as PGLA, PEG, PGLA and base polymer. Materials further include textiles such as rayon, nylon, silk, Kyeon, Kevlar, and cotton. Materials also include biopolymers such as collagen, filaments, and coated polymeric material. Materials further include elastomers such as urethanes, silicones, nitrites, Teco Flux, carbothane, and silicone hybrids

The textile materials may be knits or woven and may be expandable. The textiles include polybutester such as Novatyil, PGA (Dexon), PLA (polylactic acid), polyglactin acid (Vicryl), polydiaxanone (POS) and polylyconate (Maxon).

Pusher materials for the proximal shaft include Grilamids, nylon (12 30% glass (PARG)), polyamide, filled HDPE, polybutylene terephthalate, rigid polyurethane and polypropylene, that is 30% glass filled.

The aneurysm filler device embodiments may be formed by one or more processes such as reflow, thermal welding, adhesive welding, extrusion processing or other mechanisms for attaching two or more materials together.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

What is claimed is:

1. An aneurysm filler comprising: a main body comprising a pusher portion, a filler portion and a bridge portion, wherein the bridge portion contacts both the pusher portion and the filler portion.

2. The aneurysm filler of claim 1, wherein the bridge contact to the filler portion is a thermal weld or an adhesive weld, or an extruded contact.

3. The aneurysm filler of claim 1, wherein the bridge contact to the pusher portion is a thermal weld, or an adhesive weld or an extruded contact.

4. The aneurysm filler of claim 1, wherein the bridge is positioned in a manner effective to render the detacher movable in multiple directions.

5. An aneurysm filler comprising: a main body comprising: a first pusher portion, a second pusher portion and a bridge portion that contacts both the first and second pusher portion.

6. The aneurysm filler of claim 5, further comprising a second bridge and a filler portion, wherein the second bridge contacts the second pusher portion and the filler portion.

7. An aneurysm filler comprising: a main body comprising a plurality of bridges and pusher portions and filler portions, wherein the bridges are positioned between the pusher portions and filler portions in a manner effective to increase the movability of the aneurysm filler detacher.
